(19) United States
(12) Patent Application Publication
(10) Pub. No.: US 2008/0254086 A1
(43) Pub. Date: Oct. 16, 2008

(54) Controlled Release Compositions

(76) Inventors: James E. Brown, Los Gatos, CA (US); John W. Gibson, Springville, AL (US)

Correspondence Address: DURECT CORPORATION;THOMAS P. MCCRACKEN, 2 RESULTS WAY, CUPERTINO, CA, 95014, US

(21) Appl. No.: 11/793,908

(22) PCT Filed: Dec. 23, 2004
(86) PCT No.: PCT/US04/43079

§ 371 Date: Mar. 31, 2008

(51) Int. Cl. A61F 2/00; A61K 38/08

(52) U.S. Cl. 424/422; 514/15

(57) ABSTRACT

Controlled release compositions for controlling release of a GnRH molecule or a GnRH analog are provided. The compositions include a GnRH molecule or GnRH analog as an active agent, and a controlled release component for controlling release of the GnRH molecule or GnRH analog from the composition. The compositions provide a sustained mean steady state plasma concentration (Cₛₛ) of the active agent of at least about 1.5 ng/mL for a period of at least about 48 hours when the composition is administered to a subject. In addition, the use of a controlled release component in the manufacture of a composition for the controlled release of a GnRH molecule or GnRH analog is provided. The controlled release component is capable of providing a sustained mean steady state plasma concentration (Cₛₛ) of the active agent of at least about 1.5 ng/mL for a period of at least about 48 hours when the manufactured composition is administered to a subject. The controlled release component can include a polymeric material and/or a non-polymeric material. When the compositions are administered to a subject, for example when implanted, the compositions release the active agent in a controlled fashion. Methods for producing the compositions are also provided, as are methods of using the compositions to provide for controlled release of the GnRH molecule or GnRH analog in a subject.

### TECHNICAL FIELD

The present invention is generally in the field of controlled release compositions for delivery of peptide or protein biopharmaceuticals, in particular GnRH or GnRH analog biopharmaceuticals.

### BACKGROUND OF THE INVENTION

Biodegradable controlled release systems for active agents are well known in the art. Biodegradable matrices for drug delivery are useful because they obviate the need to remove the drug-depleted device.

The most common matrix materials used for controlled release systems are polymers. The field of biodegradable polymers has developed rapidly since the synthesis and biodegradability of polylactic acid was reported by Kulkarni et al. (1966) Arch. Surg. 93:839. Examples of other polymers which have been reported as useful as a matrix material for controlled release systems include polyanhydrides, polyesters such as polyglycolides and polylactide-co-glycolides, polyamino acids such as polylysine, polymers and copolymers of polyethylene oxide, acrylic terminated polyethylene oxide, polyamides, polyarethanes, polyorthoesters, polyacrylonitriles, and polyphosphazenes. See, e.g., U.S. Pat. Nos. 4,891,225 and 4,906,474 to Langer (polyanhydrides), 4,767,628 to Hutchinson (polylactide, polylactide-co-glycolide acid), 4,530,840 to Tice, et al. (polylactide, polyglycolide, and copolymers), and 5,234,520 (Dunn et al., biodegradable polymers for controlled delivery in treating periodontal disease).

Degradable materials of biological origin are well known including, for example, crosslinked gelatin. Hyaluronic acid has been crosslinked and used as a degradable swelling polymer for biomedical applications (see, e.g., U.S. Pat. No. 4,957,744 and Della Valle et al. (1991) Polym. Mater. Sci. Eng., 62:731-735).

Biodegradable hydrogels have also been developed for use in controlled delivery systems and serve as carriers of biologically active materials such as hormones, enzymes, antibiotics, antineoplastic agents, and cell suspensions. See, e.g., U.S. Pat. No. 5,149,543. In addition, dispersion systems are also currently in use as carriers of substances, particularly biologically active compounds. Dispersion systems used for pharmaceutical and cosmetic formulations can be categorized as either suspensions or emulsions. Suspensions are comprised of solid particles ranging in size from a few nanometers up to hundreds of microns, dispersed in a liquid medium using suspending agents. Solid particles include microparticles, microcapsules, and the like. Emulsions are generally dispersions of one liquid in another stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, for example, those described in U.S. Pat. Nos. 4,622,219 and 4,725,442. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

A number of systems for forming an implant in situ have been described. For example, U.S. Pat. No. 4,938,763 describes a method for forming an implant by dissolving a non-reactive, water insoluble thermoplastic polymer in a biocompatible, water-soluble solvent to form a liquid, placing the liquid within the body, and allowing the solvent to dissipate to produce a solid implant. The polymer solution can be placed in the body via syringe. The implant can assume the shape of its surrounding cavity. Alternatively, an implant can be formed from reactive, liquid oligomeric polymers which contain no solvent and which cure in place to form solids, usually with the addition of a curing catalyst.

### SUMMARY OF THE INVENTION

Controlled release compositions for controlling release of a GnRH molecule or a GnRH analog are provided. It is thus an object of the invention to provide a controlled release composition comprising a GnRH molecule or GnRH analog and a controlled release component for controlling release of the GnRH molecule or GnRH analog from the composition. The composition is capable of providing a sustained mean steady state plasma concentration (Cₛₛ) of the GnRH molecule or GnRH analog of at least about 1.5 ng/mL for a period of at least about 48 hours when the composition is administered to a subject.

It is more particularly an object of the present invention to provide a composition suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts. In this regard, the compositions of the present invention can be used to establish a sustained mean Cₛₛ of the GnRH molecule or GnRH analog on the order of at least about 1.5 ng/mL for a period of at least about 48 hours when the composition is administered to a subject, in some compositions, a sustained mean Cₛₛ of the GnRH molecule or GnRH analog on the order of at least about 2.0 ng/mL or more can be established, in other compositions at least about 2.5 ng/mL or more, and in yet further compositions, at least about 3.0 to 5.0 ng/mL or more. All of the novel compositions of the present invention are capable of providing these high plasma levels for a period of at least about 48 hours in the subject after administration, in some compositions, these levels can be established for a period of at least about a week or more or at least about 2 weeks or more, and in yet further compositions these plasma levels are established for a period of at least about a month or more.

It is another object of the invention to provide for the use of a controlled release component in the manufacture of a composition for the controlled release of a GnRH molecule or GnRH analog. The controlled release component is capable of providing a sustained mean steady state plasma concentration (Cₛₛ) of the GnRH molecule or GnRH analog of at least about 1.5 ng/mL for a period of at least about 48 hours when the composition is administered to a subject.

Here again, it is more particularly an object of the present invention to provide for the use of a controlled release component in the manufacture of a composition suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts. Thus, the controlled release components can be used to produce compositions capable of establishing a sustained mean Cₛₛ of the GnRH molecule or GnRH analog on the order of at least about 1.5 ng/mL for a period of at least about 48 hours when the composition is administered to a subject, in some uses, the compositions so produced can be used to establish a sustained mean Cₛₛ of the GnRH molecule or GnRH analog on the order of at least about 2.0 ng/mL or more, in other uses the compositions can be used to establish a mean Cₛₛ of at least about 2.5 ng/mL or more, and yet further compositions can be produced to establish a mean Cₛₛ of at least about 3.0 to 5.0 ng/mL or more. In the practice of the invention, controlled release components can be used to produce composition capable of providing these high plasma levels for a period of at least about 48 hours in the subject after administration, in other compositions, the levels can be established for a period of at least about a week or more or at least about 2 weeks or more, and in yet further compositions these plasma levels are established for a period of at least about a month or more.

The compositions of the present invention can be provided in any suitable dosage form depending upon the manner in which the composition will be administered. In this regard, the present compositions may be provided as oral dosage forms and administered by oral routes (e.g., administered as capsules including hard capsules and soft capsules, solid preparations such as granules, tablets, pills, troches or lozenges, cachets, pellets, powders, particulates, microparticulates (and any other particulate form). Alternatively, the present compositions can be provided in dosage forms suitable for administration by non-oral routes (e.g., any parenteral route such as IM (intramuscular), subcutaneous, transdermal, visceral, IV (intravenous), IP (intraperitoneal), intraarterial, intrathecal, intratumoral, perivascular, intracranial, periophthalmic, intrabladder, intravaginal, intraurethral, intrarectal, and adventitial routes, as well as other suitable dosage forms).

In certain aspects of the invention, the compositions are intended for administration by implantation, and can thus be provided in a shaped solid dosage form such as a sphere, rod, slab, film, fiber, needle, cylinder, sheet, tube, particle, or any other suitable geometry including microparticles, microspheres, and/or microcapsules. The compositions can further be provided in any suitable size and shape of implantable device for specialized locations, for example as a uterine implant, periurethral implant, splint, or stent (formed from, containing, or coated with the composition).

Compositions provided as solid dosage forms suitable for implantation can be implanted at a desired site surgically, or using minimally invasive techniques employing trocars, catheters, etc. The implantable dosage forms can thus be implanted into suitable tissues using standard techniques, such as where the dosage forms are implanted intradermally, subdermally, subcutaneously, intraperitoneally, intramuscularly, or intralumenally (e.g., intraarterially, intravenously, intravaginally, or even rectally). The solid dosage forms can alternatively be fabricated as part of a matrix, graft, prosthetic or coating. If an implantable dosage form is manufactured in particulate form, e.g., as a microparticle, microsphere or microcapsule, it can then be implanted into suitable tissue using a cannula, needle and syringe or like instrument to inject a suspension of the particles.

In certain other aspects of the invention, the compositions are intended for administration by implantation, yet are provided in a dosage form that is injectable and suitable for forming either a depot or a solid or semi-solid implant in situ upon or after administration. In this regard, the dosage form can be provided as either a fluid or liquid composition, or as a solid or semi-solid composition that can be rendered into a fluid or liquid form by way of addition of suitable solvents and/or plasticizers. These implantable dosage forms can be provided as an emulsion, a paste, a gel, a slurry or a liquid. In certain compositions, one or more solvents/plasticizers added to or present in the composition are capable of dissipating, diffusing or leaching away from the composition upon placement within a biological system, whereby the remaining composition can then coagulate or precipitate to form a depot, semi-solid or solid implant in situ.

With regard to any of the compositions of the present invention that are provided in a dosage form suitable for administration by implantation, the active agent (the GnRH molecule or GnRH analog) can be generally mixed with the controlled release component to provide a substantially homogeneous composition (e.g., the GnRH molecule or GnRH analog is distributed uniformly within the controlled release component such as in a monolithic implant dosage form), or the active agent can be coated with the controlled release component and provided as a coated solid such as a rod, a coaxial rod, a particle, sphere or microsphere dosage form.

In certain aspects of the invention, the composition is provided and administered as a single dosage form. For example, the composition can be provided as an implantable solid dosage form such as a rod of fiber. In other aspects, the composition is provided and administered as a plurality of dosage forms. For example, the compositions of the invention can be provided as a combination of an implantable solid dosage form and an injectable depot. In certain aspects, the composition is provided as a single dosage form that is administered as a single dosage unit, that is, a single dosage form is used to provide the recited sustained mean steady state plasma concentrations of the GnRH molecule or GnRH analog. For example, a single solid implantable dosage form such as a rod or fiber can be administered to a subject to provide the desired pharmacokinetics of the present invention. In other instances, multiple dosage units of a single dosage form can be administered to provide the recited sustained mean steady state plasma concentrations of the GnRH molecule or GnRH analog, such as wherein a plurality (two or more) of solid implantable dosage forms are administered either simultaneously, concurrently, or sequentially to provide the desired pharmacokinetics of the present invention. In yet further aspects of the invention, multiple dosage forms, each representing a single dosage unit, can be administered either simultaneously, concurrently, or sequentially to provide the desired pharmacokinetics of the present invention. Whenever multiple dosage forms and/or units are administered, the actual dose of the GnRH molecule or GnRH analog in each form or unit can be the same or different. In this way, any desired sustained mean steady state plasma concentration of the GnRH molecule or GnRH analog can be achieved in a given subject by way of administering a single dosage form and/or dosage unit of sufficient dose, or by combining a plurality of dosage forms and/or units containing the same or different dose of the GnRH molecule or GnRH analog to tailor a specific dose sufficient to establish the desired plasma concentration in a given subject.

In certain aspects of the invention, the controlled release component used to produce the controlled release composition comprises a polymer material, that is, the controlled release component either contains a polymer material or is comprised substantially of a polymer material. In a certain compositions, the controlled release component comprises a polymer selected from the group consisting of polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polycaprolactone, polyesteramides, polyphosphazines, polycarbonates, polyamides, and copolymers thereof. In a particular composition, the controlled release component comprises a polymer that is an AB copolymer wherein the A component is a copolymer of lactide, glycolide, or caprolactone, and the B component is a polyalkyleneglycol.

In certain other aspects of the invention, the controlled release component used to produce the controlled release composition comprises a non-polymer material, that is, the controlled release component either contains a non-polymer material or is comprised substantially of a non-polymer material. In certain compositions, the controlled release component comprises a non-polymeric material that is substantially insoluble in water or in an aqueous biological system. In such cases, the composition may further contain a solvent that is dispersible, soluble or miscible in water or in an aqueous system. The solvent may thus be an organic solvent that is capable of dissipating, diffusing or leaching away from the composition upon placement within a biological system, whereby the carrier can then coagulate or precipitate to form a solid implant in situ.

In yet another aspect of the invention, the non-polymeric material is a liquid carrier material, preferably a high viscosity liquid carrier material (“HVLCM”) having a viscosity of at least about 5,000 cP at 37° C. and which does not crystallize neat under ambient or physiological conditions. Such liquid carrier materials can be combined with a solvent in which the carrier material is soluble. If a HVLCM is used, it is preferred that the solvent is sufficient to lower the viscosity of the HVLCM. In certain compositions a further material is included that is immiscible with the non-polymeric material, for example where the composition is an emulsion. In these compositions, the non-polymeric material may be present in either the dispersed or the continuous phase of the emulsion.

In each of the compositions of the present invention, the GnRH molecule or GnRH analog active agent can be present in an amount of at least about 10 wt % relative to the total weight of the composition. In other compositions, the active agent is present in an amount of at least about 15 wt %, 20 wt %, 25 wt % or 30 wt % relative to the total weight of the composition, or more. In certain aspects of the invention, the total amount of the GnRH molecule or GnRH analog in the composition (whether as single or multiple dosage forms and/or units) is between about 1 and 50 mg, in other compositions, between about 1.5 and 40 mg, and in still others between about 2 and 40 mg, 3 and 35 mg, or between about 5 and 20 mg. In certain compositions, the active agent is a GnRH analogue such as desorelin, tryptorelin, goserelin, and leuprolide.

In certain aspects of the invention, it may be desirable that the controlled release composition is constructed such that the GnRH molecule or GnRH analog active agent is released from the composition without a significant or substantial initial burst. In this regard, certain compositions can be provided wherein less than about 50% of the initial dose of the active agent is released from the composition within about 24 to 48 hours of administration to the subject, in other compositions, less than about 40% is released in this initial period, in still others, less than about 30% is released. In certain other compositions of the invention, the GnRH molecule or GnRH analog active agent is released from the composition without a substantial lag period or with a minimal lag period. In these same, or in other compositions, the active agent is released in a controlled manner suitable to provide for zero order or linear release kinetics.

It is a further object of the invention to provide a method for establishing therapeutically effective plasma levels of a GnRH molecule or GnRH analog for about 48 hours or more in a subject. The method entails administering any one of the above-described controlled release compositions to the subject such that, after administration, the composition provides a sustained mean steady state plasma concentration (Cₛₛ) of the GnRH molecule or GnRH analog of at least about 1.5 ng/mL for a period of at least about 48 hours in the subject.

It is more particularly an object of the present invention to provide a method suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts. Accordingly, the method of the present invention can be used to establish a sustained mean Cₛₛ of the GnRH molecule or GnRH analog on the order of at least about 1.5 ng/mL for a period of at least about 48 hours after the composition is administered to a subject, in some particular methods, a sustained mean Cₛₛ of the GnRH molecule or GnRH analog on the order of at least about 2.0 ng/mL or more can be established, in other methods at least about 2.5 ng/mL or more, and in yet further methods, at least about 3.0 to 5.0 ng/mL or more. All of the novel methods of the present invention are capable of providing these high plasma levels for a period of at least about 48 hours in the subject after administration. In some compositions, these levels can be established for a period of at least about a week or more or at least about 2 weeks or more, and in yet further compositions these plasma levels are established for a period of at least about a month or more.

In the methods of the invention, the controlled release composition can be administered using any suitable procedure. Depending upon the selected dosage form(s) and the selected site(s) of administration, the compositions can be delivered or implanted using minimally invasive procedures at a site where release is desired. These procedures can include implantation using trocars or catheters, injection using standard needle and syringes (of, e.g., powders, particles, microparticles, microspheres, microcapsules), ingrafting or surgical or non-surgical placement (of, e.g., a matrix, graft, prosthetic or coating), and the like. The compositions are designed so that the GnRH molecule or GnRH analog active agent is released in the desired dosage over a defined period of time, and achieves the desired sustained mean Cₛₛ for the desired period. In some methods, the compositions can be manufactured using suitable controlled release components so that they degrade during and/or after release of the active agent is achieved.

In one preferred method, the composition is formulated to include a GnRH molecule or GnRH analogue in a solid implant form. The composition is then administered to a subject in order achieve the target steady state plasma level, and thereby exert an effect upon the production, function, or activity of a gonadotrophin (LH or FSH) in the subject.

It is an advantage of the present invention that the controlled release compositions are able to establish a sustained mean Cₛₛ of the GnRH molecule or GnRH analog on the order of at least about 1.5 ng/mL for a period of at least about 48 hours after the composition is administered to a subject. It is a further advantage of the invention that the compositions are readily constructed to provide any number of different pharmaceutical forms, and further to provide a wide range of different pharmacological release characteristics depending upon the intended site of administration and medical application.

These and other objects, aspects and advantages of the present invention will readily occur to the skilled practitioner upon reading the instant disclosure, specification and claims.

### DETAILED DESCRIPTION OF THE PREFERRED EMBODIMENTS

Before describing the present invention in detail, it is to be understood that this invention is not limited to particularly exemplified controlled release component or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise. Thus, for example, reference to “a controlled release component” includes a mixture of two or more such components, reference to “an agent” or “a GnRH active agent” includes mixtures of two or more such agents, and the like. In addition, whenever a specified range is provided in the instant specification and claims, use of the modifier “about” is applied to all values or quantities specified by that range. Thus, the phrase “about 1 to 50 mg” means “about 1 to about 50 mg”, and the phrase “about 3.0 to 5.0 ng/mL” means “about 3.0 to about 5.0 ng/mL”, and the like.

It is an object of the present invention to provide a controlled release composition comprising a GnRH molecule or GnRH analog as an active agent and a controlled release component for controlling release of the active agent from the composition. The composition is capable of providing a sustained mean steady state plasma concentration (Cₛₛ) of the GnRH molecule or GnRH analog of at least about 1.5 ng/mL for a period of at least about 48 hours when the composition is administered to a subject.

It is more particularly an object of the present invention to provide a composition suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts.

It is another object of the invention to provide for the use of a controlled release component in the manufacture of a composition for the controlled release of a GnRH molecule or GnRH analog. The controlled release component is capable of providing a sustained mean steady state plasma concentration (Cₛₛ) of the GnRH molecule or GnRH analog of at least about 1.5 ng/mL for a period of at least about 48 hours when the composition is administered to a subject.

Here again, it is more particularly an object of the present invention to provide for the use of a controlled release component in the manufacture of a composition suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts.

It is a still further object of the invention to provide a method for establishing therapeutically effective plasma levels of a GnRH molecule or GnRH analog for about 48 hours or more in a subject. The method entails administering any one of the above-described controlled release compositions to the subject such that, after administration, the administered composition provides a sustained mean steady state plasma concentration (Cₛₛ) of the GnRH molecule or GnRH analog of at least about 1.5 ng/mL for a period of at least about 48 hours in the subject.

Again, it is more particularly an object of the present invention to provide a method suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts.

A number of controlled release compositions for use in long-term, or controlled delivery of a GnRH molecule or GnRH analog (a “GnRH active”) are currently available. The vast majority of such compositions employ a biodegradable, implantable polymer system as the controlled release component, wherein the composition is a solid dosage form such as a shaped implant, or a depot of particles.

GnRH is of central importance to the regulation of fertility. In males and females, GnRH is released from the hypothalamus into the bloodstream and travels via the blood to the pituitary, where it induces the release of the gonadotropins, luteinizing hormone (“LH”) and follicle stimulating hormone (“FSH”) by gonadotroph cells, and regulates androgens, estrogens, and progestins. The key feature of GnRH secretion is pulsatile release, with the frequency or amplitude of GnRH pulses controlling whether FSH and/or LH are secreted, and the relative amounts that are secreted. An important mechanism of action for GnRH analogs (agonists) is the loss of GnRH receptors (due to desensitization) in the plasma membrane of gonadotropes, and the natural down-regulation in response to prolonged occupancy of the receptors by the GnRH agonist.

GnRH agonist medicaments have been used to treat a variety of diseases and conditions, e.g., to treat hormone-dependent cancers (such as prostate cancer); to treat endometriosis; to treat early puberty, to control estrogen production; to treat fertility conditions; and the like. Some common names and tradenames for commercial GnRH agonist products include leuprolide (trade name: Lupron®, Abbott/TAP; Viadur®, Alza), goserelin (trade name: Zoladex®; Zeneca), buserelin (Hoechst), triptorelin (also known as Decapeptyl, D-Trp-6-LHRH and Debiopharm.®.; Ipsen/Beaufour), nafarelin (trade name Synarel®; Syntex), lutrelin (Wyeth), cystorelin (Hoechst), gonadorelin (Ayerst) and histrelin (Ortho), luliberin, desorelin, avorelin, cetrrelix, teverelix, ramorelix, ganirelix, antide, nictide, and azaline.

The most common GnRH analogue medicaments are implantable controlled-release formulations based on leuprolide or goserelin, where the implants are used to provide 1- to 3-month therapeutic levels of the GnRH active agent in the treatment of prostate cancers or endometriosis. Leuprolide is a generic drug. Lupron contains a water-soluble salt form of the GnRH active (leuprolide acetate) encapsulated by a biodegradable polymer carrier (polylacticacid “PLA”) to form microspheres. The microspheres are freeze-dried, and then administered IM to provide a controlled release depot implant. Zoladex also contains a water-soluble salt form of the GnRH active agent (goserelin acetate), however the active agent is dispersed within a biodegradable polymer matrix (D, L-lactic and glycolic acid copolymer “PLGA”) and extruded to form a solid controlled release implant.

Administration of the Lupron or Zoladex controlled release compositions results in the following general pharmacokinetics: upon administration, there is typically an initial burst phase, wherein a large amount of the GnRH active is released to provide a maximum plasma concentration (Cₘₐₓ) within the first 24 to 48 hours of administration; followed by a steady state phase wherein release of the GnRH active is at least partially constant and sufficient to provide a steady state plasma concentration (Cₛₛ) for a period of from weeks to several months; followed by a tailing off of plasma concentrations. The sustained mean steady state plasma concentration (Cₛₛ) of the GnRH molecule or GnRH analog attained from administration of Lupron during the period of about 2 to 16 weeks typically ranges between about 0.2 to 1.0 ng/mL, and typically around 0.5 ng/mL from the commercial dosage forms that are administered at 7.5, 22.5 and 30 mg doses. The amount of the GnRH active lost from the Lupron implant during the initial burst is substantial, in some cases approaching up to 50% of the total initial GnRH active dosage provided. The sustained mean steady state plasma concentration (Cₛₛ) of the GnRH molecule or GnRH analog attained from administration of Zoladex during the same period is also typically around 0.5 ng/mL. These low steady state plasma concentrations are generally considered adequate for common therapies such as treatment of prostate cancers.

Release of the GnRH actives from the above-described commercially available controlled release compositions can occur with lags, bursts and other characteristics, that prevent such dosage forms from achieving a substantially constant, zero or first order release profile. This is because the GnRH actives are generally not soluble in common hydrophobic polymer controlled release materials such as DL-polylactide-co-glycolide (“DL-PLG”), and as such must be provided as two-phase compositions in which the minor component (e.g. the GnRH active) exists as a dispersed phase within the major component (e.g. the DL-PLG). Due to various physical and chemical properties of the controlled release component, the release of the GnRH active from DL-PLGs typically does not occur by simple diffusion through the polymer matrix. Rather, release occurs by diffusion through aqueous channels that form when the composition is placed into an aqueous environment.

Release of the GnRH active from the controlled release composition thus occurs most usually by diffusion through the aqueous channels formed by hydration of the polymer. The resulting release profile tends to be biphasic in which two periods of release are separated by a period during which little or no peptide release occurs. The “dead” period that occurs between the two release phases is particularly problematic for the GnRH actives, where therapeutic objectives are typically continuous suppression of one or more gonadotrophic hormone.

One approach to minimize or eliminate the “dead” period involves increasing the peptide content of the composition. As the peptide content of the composition is increased, inter-particle contact between the peptide particles increases, providing a more extensive network of pores, and the proportion of peptide that is released during the initial phase increases, in a so-called initial burst phase, ultimately consuming a substantial amount, if not all of the GnRH originally provided in the composition. Release typically follows the well-known Higuchi model for release from a dispersed-drug monolithic device and exhibits square-root-of-time kinetics.

Another approach to minimizing the dead period and achieving a more constant release of drug involves the use of polymer compositions that degrade relatively rapidly. For example, U.S. Pat. Nos. 4,767,628, 5,004,602, 5,366,734 to Hutchinson describe continuous release compositions for a GnRH active, wherein the initial diffusion-controlled phase of release and the second degradation-controlled phase of release are made to overlap by careful choice of the monomer ratio and the molecular weight of the DL-PLG.

Still another approach involves the use of biodegradable hydrogels so that the permeability of the peptide (e.g., the GnRH active) in the polymer matrix is significantly increased. For example, U.S. Pat. Nos. 4,526,938 and 4,942,035 to Churchill describe continuous release compositions comprising a GnRH active and an amphipathic block copolymer in which the hydrophobic component is biodegradable and the hydrophilic component may or may not be biodegradable. Generally, these compositions contain relatively large amounts of the hydrophilic component such that the resulting polymers are hydrogels capable of absorbing large amounts of water.

U.S. Pat. No. 6,159,490 to Deghenghi describes a method for producing implants for delivery of a GnRH active (i.e., the GnRH analog leuprolide) from copolymers of lactide and glycolide for periods of from 1 to 12 months. Deghenghi's process involves a wet granulation process to combine the GnRH active with the polymer controlled release component. U.S. Pat. No. 6,217,893 to Pellet et al. describes controlled release compositions containing a GnRH active, using polymer or copolymer controlled release components (lactide and glycolide having a hydrophilic character. No examples of the preparation of or release from implants are given.

Although these and other approaches to producing controlled release compositions for a GnRH active may have been sufficient to reduce erratic or widely variable release kinetics, the art has heretofore not considered how to produce a composition in accordance with the present invention, that is, a composition capable of providing substantially higher sustained mean steady state plasma concentrations (Cₛₛ) of the GnRH molecule or GnRH analog in the range of the compositions of the present invention wherein it is desired to establish steady state plasma concentrations of at least about 1.5 ng/mL for a period of at least about 48 hours in the subject, in some cases on the order of at least about 2.0 ng/mL or more, in others at least about 2.5 ng/mL or more, and yet further cases at least about 3.0 to 5.0 ng/mL or more. Furthermore, the art has heretofore not considered how to produce a composition in accordance with the present invention that is capable of providing these novel high plasma levels for a period of at least about 48 hours in the subject after administration, in other cases for a period of at least about a week or more or at least about 2 weeks or more, and in yet further cases these novel high plasma levels can be established for a period of at least about a month or more.

In addition, the art has heretofore not considered how to produce a composition in accordance with the present invention (capable of providing these novel high plasma levels for the recited periods), wherein the composition further serves to reduce or eliminate highly variable release kinetics during steady state conditions, for example, compositions that release the GnRH active agent at a high level over a prolonged period of time, and that provide more controlled zero-order or linear release kinetics rather than biphasic release kinetics.

In this regard, compositions produced in accordance with the present invention can provide a high sustained mean steady state plasma concentrations (Cₛₛ) of the GnRH molecule or GnRH analog, and further enable a more constant or linear rate of release of the active agent. Such compositions can be provided as a monolithic implant prepared with a hydrolytically biodegradable hydrophobic polymer such as poly (DL-lactide-co-glycolide), DL-PLG, which incorporates a small amount of hydrophilic polymer. The use of hydrophobic polymers such as PLGs with incorporation of small amounts of hydrophilic polymer such as poly (ethylene glycol), PEG, preferably covalently linked into the hydrophobic polymer backbone provides particularly beneficial release profiles. In addition, the combination of such material choices with a simple process involving, for example, dry blending, compounding (first-pass extrusion), grinding, and re-extrusion, can further provide for beneficial release profiles. The monolithic implant compositions can be any shaped article such as a rod, needle, film, sphere, cylinder, sheet, or other geometry including microparticles, microspheres, and/or microcapsules. A preferred manufacturing process avoids the use of solvent to mix the polymeric controlled release component with the GnRH active. The exemplary composition is designed to provide monophasic release, i.e., where release is typically linear or zero order, but may include continuous release where the initial “burst” or “lag” effect is minimal or not present.

The phrase “without an initial burst,” as used herein, intends that the particular controlled release composition being referred to does not release a substantial amount of the GnRH active from the composition upon normal administration that becomes pharmacologically available in an appreciable amount during a predetermined initial period. The presence and level of an initial burst of a GnRH agent from a given composition can be readily determined by the skilled artisan employing standard pharmacological testing techniques well known in the art. Suitable in vitro burst release characterization methods include the USP II Paddle Method, using standard buffer, mixing and heat conditions. The burst release characteristics of a given composition can also readily be determined using standard in vivo testing, such as by monitoring plasma concentrations of the GnRH agent in an animal subject, over a given time period. In the compositions of the present invention, preferably less than about 40 to 60% of the GnRH agent is released within the first 24 to 48 hours after administration, more preferably less than about 30 to 50%, and even more preferably less than about 20 to 40% is released within this initial time period.

I. Materials and Compositions

A. GnRH Active Agents

Essentially any GnRH active agent can be combined with a suitable controlled release component to form a composition (and subsequent dosage form) according to the present invention using conventional processes including those methods described herein. Accordingly, as used herein a “GnRH active” or “GnRH active agent” can include any GnRH molecule or GnRH analog which, when administered to an organism (human or animal subject) induces a desired pharmacologic and/or physiologic effect by local and/or systemic action. The GnRH active is typically referred to as a peptide or protein biopharmaceutical. As used herein, the term “protein” includes peptides, polypeptides, consensus molecules, analogs, derivatives or combinations thereof. The term thus encompasses recombinant or naturally occurring molecules, whether human or animal in origin, including naturally occurring, synthetic, semi-synthetic or recombinantly produced GnRH molecules or GnRH analogs.

As used herein, the term “GnRH analog” is intended to encompass peptidic compounds that mimic the structure of luteinizing hormone releasing hormone. A GnRH analog may be a GnRH agonist.

As used herein, a “GnRH agonist” is intended to refer to a compound that stimulates the GnRH receptor such that release of luteinizing hormone and/or FSH is stimulated. Examples of GnRH agonists include leuprolide (trade name: Lupron®, Abbott/TAP; Viadur®, Alza), goserelin (trade name: Zoladex®; Zeneca), buserelin (Hoechst), triptorelin (also known as Decapeptyl, D-Trp-6-LHRH and Debiopharm.®.; Ipsen/Beaufour), nafarelin (trade name Synarel®; Syntex), lutrelin (Wyeth), cystorelin (Hoechst), gonadorelin (Ayerst) and histrelin (Ortho), luliberin, desorelin, avorelin, cetrrelix, teverelix, ramorelix, ganirelix, antide, nictide, and azaline. Leuprolide agonists are particularly preferred for use in the compositions of the present invention.

In the practice of the invention, the GnRH active is combined with a controlled release component to form a controlled release composition.

B. Polymer Controlled Release Components

The compositions disclosed herein can be produced using a variety of biocompatible and biodegradable polymer controlled release components. “Biodegradable”, as defined herein, means the polymer will degrade or erode in vivo to form smaller chemical species, wherein the degradation can result, for example, from enzymatic, chemical, and physical processes. In certain preferred compositions, the polymer controlled release component is substantially hydrophobic and degrades by hydrolysis. The term “biocompatible” is used herein to refer to a polymer and any degradation products of the polymer that present no significant, deleterious or untoward effects on the recipient's, that is, the subject's body.

Examples of biodegradable polymers and oligomers suitable for use in the compositions and methods of the present invention include, but are not limited to: poly(lactide)s; poly(glycolide)s; poly(lactide-co-glycolide)s; poly(lactic acid)s; poly(glycolic acid)s; and poly(lactic acid-co-glycolic acid)s; poly(caprolactone)s; poly(malic acid)s; polyamides; polyanhydrides; polyamino acids; polyorthoesters; polyetheresters; polycyanoacrylates; polyphosphazines; polyphosphoesters; polyesteramides; polydioxanones; polyacetals; polyketals; polycarbonates; polyorthocarbonates; degradable polyurethanes; polyhydroxybutyrates; polyhydroxyvalerates; polyalkylene oxalates; polyalkylene succinates; chitins; chitosans; oxidized celluloses; and copolymers, terpolymers, blends, combinations or mixtures of any of the above materials.

As used herein, “hydrophobic” refers to a polymer that is substantially not soluble in water. As used herein, “hydrophilic” refers to a polymer that may be water-soluble or to a polymer having affinity for absorbing water, but typically not when covalently linked to the hydrophobic component as a co-polymer, and which attracts water.

Hydrophilic polymers suitable for use herein can be obtained from various commercial, natural or synthetic sources well known in the art. Suitable hydrophilic polymers include, but are not limited to: polyanions including anionic polysaccharides such as alginate; agarose; heparin; polyacrylic acid salts; polymethacrylic acid salts; ethylene maleic anhydride copolymer (half ester); carboxymethyl amylose; carboxymethyl cellulose; carboxymethyl dextran; carboxymethyl starch; carboxymethyl chitin/chitosan; carboxy cellulose; 2,3-dicarboxycellulose; tricarboxycellulose; carboxy gum arabic; carboxy carrageenan; carboxy pectin; carboxy tragacanth gum; carboxy xanthan gum; carboxy guar gum; carboxy starch; pentosan polysulfate; curdlan; inositol hexasulfate; beta.-cyclodextrin sulfate; hyaluronic acid; chondroitin-6-sulfate; dermatan sulfate; dextran sulfate; heparin sulfate; carrageenan; polygalacturonate; polyphosphate; polyaldehydro-carbonic acid; poly-1-hydroxy-1-sulfonate-propen-2; copolystyrene maleic acid; mesoglycan; sulfopropylated polyvinyl alcohols; cellulose sulfate; protamine sulfate; phospho guar gum; polyglutamic acid; polyaspartic acid; polyamino acids; and any derivatives or combinations thereof. One skilled in the art will appreciate other hydrophilic polymers that are also within the scope of the present invention.

Various water-soluble polymers suitable for use herein include, but are not limited to: poly (alkyleneglycol), polyethylene glycol (“PEG”); propylene glycol; ethylene glycol/propylene glycol copolymers; carboxylmethylcellulose; dextran; polyvinyl alcohol (“PVOH”); polyvinyl pyrolidone; poly (alkyleneamine)s; poly (alkyleneoxide)s; poly-1,3-dioxolane; poly-1,3,6-trioxane; ethylene/maleic anhydride copolymers; polyaminoacids; poly (n-vinyl pyrolidone); polypropylene oxide/ethylene oxide copolymers; polyoxyethylated polyols; polyvinyl alcohol succinate; glycerine; ethylene oxides; propylene oxides; poloxamers; alkoxylated copolymers; water soluble polyanions; and any derivatives or combinations thereof. In addition, the water-soluble polymer may be of any suitable molecular weight, and may be branched or unbranched.

In certain contemplated compositions of the invention, a hydrophobic polymer component is co-polymerized with a hydrophilic polymer, or monomers, to yield a polymeric controlled release system, most preferably a block copolymer, or blended with a hydrophilic polymer to yield a blended polymeric controlled release system. These resultant polymer systems are characterized as having a small amount of hydrophilic character, but they will not form a hydrogel following immersion in an aqueous system. For example, certain polymer systems for use in the compositions of the present invention may contain a water-soluble polymer such as polyethylene glycol (PEG) in amounts typically up to 25 to 30 wt %, not imparting the hydrogel properties cited by Churchill but producing devices that exhibit monophasic or zero-order or near zero-order release kinetics. If a PEG is used in the system, the preferred molecular weight may be between about 700 Da and about 500 kDa. Other particularly preferred hydrophilic polymers for use in the polymeric controlled release systems of the invention include polyvinyl pyrolidone, polyvinyl alcohols, poly (alkyleneamine)s and poly (alkyleneoxide)s.

As used herein, “polymer” and “polymer system” include copolymers and blends unless otherwise expressly defined. Such polymeric materials can be produced using standard copolymerization techniques, such as graft copolymerisation, polycondensation and polyaddition, optionally with an appropriate catalyst. These techniques can be carried out in conventional manner well known in the polymer art as regards to time and temperature. Alternatively, the polymeric controlled release components can be produced using standard blending techniques of polymers or blending of copolymers, again carried out in conventional manner well known in the polymer art as regards to time and temperature.

The polymer controlled release component, method of manufacture, and GnRH active loading can be selected such that the composition does not form a hydrogel when contacted with or immersed in an aqueous system, for example, when a solid dosage form controlled release composition is implanted in vivo into an animal or human subject. The polymer systems used as the controlled release component are characterized by a reduced hydrophobicity relative to the pure hydrophobic polymer component by virtue of the inclusion of the hydrophilic component. This facilitates uptake of water by the composition and dissolution and release of the incorporated GnRH active agent, avoiding a lag period and leading to linear or near zero order release kinetics.

As used herein, the term “hydrogel” is used in its usual manner within the art, for example to refer to a polymer material or polymer system that swells in the presence of water or other aqueous system, shrinks in the absence or reduction of the amount of water, is able to retain a significant fraction of water within its structure, and typically does not dissolve in water. One skilled in the art will appreciate that there are a number of standard tests that one can employ in order to determine if a polymer or polymer system will act as a hydrogel, e.g., form a hydrogel, when immersed in an aqueous system such as when it is implanted in vivo into an animal or human subject.

The polymeric controlled release component and GnRH active agent may be combined with one or more additional component, for example pharmaceutically acceptable excipient materials that can act as dispersing agents, bulking agents, binders, carriers, stabilizers, glidants, antioxidants, pH adjusters, anti-irritants, and the like. The skilled artisan will appreciate that certain excipient materials can serve several of the above-referenced functions in any particular formulation. Thus, any number of suitable excipient materials can be mixed with or incorporated into the compositions of the present invention to provide bulking properties, alter GnRH active agent release rates, increase or impede water uptake, control pH, provide structural support, facilitate manufacturing processes and other uses known to those skilled in the art. The term “excipient” generally refers to a substantially inert material that is nontoxic and does not interact with other components of the composition in a deleterious manner. The proportions in which a particular excipient may be present in the composition depend upon the purpose for which the excipient is provided and the identity of the excipient.

For example, suitable excipients that can also act as stabilizers for peptides such as GnRH molecules and GnRH analogs include pharmaceutical grades of dextrose, sucrose, lactose, trehalose, mannitol, sorbitol, inositol, dextran, and the like. Such materials may thus be a saccharide such as a monosaccharide, a disaccharide, a polysaccharide or a sugar alcohol. Other suitable excipients include starch, cellulose, sodium or calcium phosphates, calcium sulfate, citric acid, tartaric acid, glycine, and combinations thereof. Examples of hydrophobic excipients that can be added to the controlled release compositions to slow hydration and dissolution kinetics include fatty acids and pharmaceutically acceptable salts thereof (e.g., magnesium stearate, steric acid, zinc stearate, palimitic acid, and sodium palitate).

It may also be useful to employ a charged lipid and/or detergent excipient in the compositions of the present invention. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics); polyoxyethylenesorbitans, e.g., TWEEN® surfactants (Atlas Chemical Industries); polysorbates; polyoxyethylene ethers, e.g. Brij; pharmaceutically acceptable fatty acid esters, e.g., lauryl sulfate and salts thereof; ampiphilic surfactants (glycerides, etc.); and like materials.

Other excipient materials can be added to the compositions to alter porosity, for example, materials like sucrose, dextrose, sodium chloride, sorbitol, lactose, polyethylene glycol, mannitol, fructose, polyvinyl pyrrolidone or appropriate combinations thereof. Additionally, the GnRH active agents may be dispersed with oils (e.g., sesame oil, corn oil, vegetable), or a mixture thereof with a phospholipid (e.g., lecitin), or medium chain fatty acid triglycerides (e.g., Miglyol 812) to provide an oily suspension.

Still further excipeint materials that can be incorporated into the compositions of the present invention include diluents of various buffer content (e.g., Tris-HCl, acetate); pH and ionic strength altering agents; additives such as antioxidants (e.g., ascorbic acid, glutathione, sodium metabisulfite); preservatives (e.g., Thimersol, benzyl alcohol, methyl paraben, propyl paraben); and dispersing agents such as water-soluble polysaccharides (e.g., mannitol, lactose, glucose, starches), hyaluronic acid, glycine, fibrin, collagen and inorganic salts (e.g., sodium chloride).

C Non-Polymer Controlled Release Components

The controlled release compositions disclosed herein can alternatively be produced using a variety of biocompatible and biodegradable non-polymer controlled release components. “Biodegradable”, as defined herein, means the non-polymer material will degrade or erode in vivo to form smaller chemical species, wherein the degradation can result, for example, from enzymatic, chemical, and physical processes. The term “biocompatible” is used herein to refer to a non-polymer material and any degradation products of that material that present no significant, deleterious or untoward effects on the recipient's, that is, the subject's body.

Selection of a suitable non-polymeric controlled release component is within the general skill in the art, using the teaching and guidance provided by the instant disclosure and specification. For example, numerous pharmaceutically acceptable non-polymeric carrier systems are available to the skilled artisan to produce liquid, spray, cream, lotion, ointment, gel, slurry, oil, emulsion, microemulsion, solid, plaster, film, particle, microparticle, powder or other suitable pharmaceutical dosage forms. These and other carrier systems are described, for example, in Remington's Pharmaceutical Sciences, 16^{th} Edition, 1980 and 17^{th} Edition, 1985, both published by Mack Publishing Company, Easton, Pa.

The controlled release compositions of the present invention may further include one or more additional component, for example pharmaceutically acceptable excipient materials that can act as dispersing agents, bulking agents, binders, carriers, stabilizers, glidants, antioxidants, pH adjusters, anti-irritants, and the like. The skilled artisan will appreciate that certain excipient materials can serve several of the above-referenced functions in any particular formulation. Thus, any number of suitable excipient materials can be mixed with or incorporated into the controlled release compositions of the present invention to provide bulking properties, alter the GnRH active agent release rates, increase or impede water uptake, control pH, provide structural support, facilitate manufacturing processes and other known uses. The proportions in which a particular excipient may be present in the composition depend upon the purpose for which the excipient is provided and the identity of the excipient.

For example, suitable excipients that can also act as stabilizers for the GnRH active agent include pharmaceutical grades of dextrose, sucrose, lactose, trehalose, mannitol, sorbitol, inositol, dextran, and the like. Such stabilizers may thus be a saccharide such as a monosaccharide, a disaccharide, a polysaccharide or a sugar alcohol. Other suitable excipients include starch, cellulose, sodium or calcium phosphates, calcium sulfate, citric acid, tartaric acid, glycine, and combinations thereof. Examples of hydrophobic excipients that can be added to slow hydration and dissolution kinetics include fatty acids and pharmaceutically acceptable salts thereof (e.g., magnesium stearate, steric acid, zinc stearate, palimitic acid, and sodium palitate).

It may also be useful to employ a charged lipid and/or detergent excipient in addition to the non-polymer controlled release component. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics); polyoxyethylenesorbitans, e.g., TWEEN® surfactants (Atlas Chemical Industries); polysorbates; polyoxyethylene ethers, e.g. Brij; pharmaceutically acceptable fatty acid esters, e.g., lauryl sulfate and salts thereof; ampiphilic surfactants (glycerides, etc.); and like materials.

Other excipient materials can be added to alter porosity of the non-polymer controlled release component, for example, materials like sucrose, dextrose, sodium chloride, sorbitol, lactose, polyethylene glycol, mannitol, fructose, polyvinyl pyrrolidone or appropriate combinations thereof. Additionally, the GnRH active may be dispersed with oils (e.g., sesame oil, corn oil, vegetable), or a mixture thereof with a phospholipid (e.g., lecitin), or medium chain fatty acid triglycerides (e.g., Miglyol 812) to provide an oily suspension.

Still further excipeint materials that can be incorporated into the compositions of the present invention include diluents of various buffer content (e.g., Tris-HCl, acetate); pH and ionic strength altering agents; additives such as antioxidants (e.g., ascorbic acid, glutathione, sodium metabisulfite); preservatives (e.g., Thimersol, benzyl alcohol, methyl paraben, propyl paraben); and dispersing agents such as water-soluble polysaccharides (e.g., mannitol, lactose, glucose, starches), hyaluronic acid, glycine, fibrin, collagen and inorganic salts (e.g., sodium chloride).

In certain embodiments of the invention, the non-polymeric controlled release component is substantially insoluble in water or in an aqueous biological system. Exemplary such non-polymeric carrier materials include, but are not limited to: sterols such as cholesterol, stigmasterol, β-sitosterol, and estradiol; cholestery esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palinitoyl, and tricosanyl spingomyelins; ceramides such as stearyl and palmitoyl ceramides; glycosphingolipids; lanolin and lanolin alcohols; and combinations and mixtures thereof. Certain preferred non-polymeric carriers include cholesterol, glyceryl monostearate, glycerol tristearate, stearic acid, stearic anhydride, glyceryl monocleate, glyceryl monolinoleate, and acetylated monoglycerides.

If one or more of the above-noted non-polymeric controlled release components is selected for use in a composition of the present invention, it will typically be combined with a compatible and suitable organic solvent for the non-polymeric material to form a composition having a consistency ranging from watery to viscous to a spreadable putty or paste. The consistency of the composition will vary according to factors such as the solubility of the non-polymeric material in the solvent, the concentration of the non-polymeric material, the concentration of the GnRH active, additives and excipients. The solubility of a non-polymeric material in a particular solvent will vary according to factors such as its crystallinity, hydrophilicity, ionic character and lipophilicity. Accordingly, the ionic character and the concentration of the non-polymeric material in the solvent can be adjusted to achieve the desired solubility. Preferred non-polymeric materials for use as the controlled release component are those that have low crystallinity, nonpolar characteristics, and are more hydrophobic.

Suitable organic solvents for use in the compositions are generally those that are biocompatible, pharmaceutically acceptable, and will at least partially dissolve the selected non-polymeric material. The organic solvent will further have a solubility in water ranging from miscible to soluble to dispersible. In certain compositions, the solvent is selected such that it is capable of diffusing, dispersing, or leaching away from the composition in situ in an aqueous system and into fluids found at the administration site, thereby forming a solid implant. Preferably, the non-polymeric material solidifies in situ to form a solid matrix within about 1-5 days after administration (implantation), preferably within about 1-3 days, preferably within about 2 hours. In addition, the solvent preferably has a Hildebrand (HLB) solubility ratio of from about 9-13 (cal/cm³)^{1/2}, and the degree of polarity of the solvent is effective to provide at least about 5% solubility in water.

Suitable organic solvents thus include, but are not limited to: substituted heterocyclic compounds such as N-methyl-2-pyrrolidone (NMP) and 2-pyrrolidone (2-pyrol); esters of carbonic acid and alkyl alcohols such as propylene carbonate, ethylene carbonate and dimethyl carbonate; fatty acids such as acetic acid, lactic acid and heptanoic acid; alkyl esters of mono-, di-, and tricarboxylic acids such as 2-ethyoxyethyl acetate, ethyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as acetone and methyl ethyl ketone; ether alcohols such as 2-ethoxyethanol, ethylene glycol dimethyl ether, glycofurol and glycerol formal; alcohols such as ethanol and propanol; polyhydroxy alcohols such as propylene glycol, polyethylene glycol (PEG), glycerin (glycerol), 1,3-butyleneglycol, and isopropylidene glycol (2,2-dimethyl-1,3-dioxolone-4-methanol); Solketal; dialkylamides such as dimethylformamide, dimethylacetamide; dimethylsulfoxide (DMSO) and dimethylsulfone; tetrahydrofuran; lactones such as ε-caprolactone and butyrolactone; cyclic alkyl amides such as caprolactam; aromatic amides such as N,N-dimethyl-m-toluamide, and 1-dodecylazacycloheptan-2-one; and the like; and mixtures and combinations thereof. Preferred solvents include N-methyl-2-pyrrolidone, 2-pyrrolidone, dimethylsulfoxide, ethyl lactate, propylene carbonate, glycofurol, glycerol formal, and isopropylidene glycol.

The organic solvent can be provided in the composition in an amount of from about 99.5 to about 1 percent by weight relative to the total weight of the composition (wt %), in an amount of from about 95 to 10 wt %, in an amount of from about 75 to 25 wt %, or in an amount of from about 60 to 40 wt %, depending upon the selected non-polymeric controlled release component, organic solvent, GnRH active, additive and/or excipient being used in the composition.

A number of suitable additives may be included with the non-polymer controlled release component in order to impart selected characteristics upon the composition. For example, the may include a minor amount of a biodegradable thermoplastic polymer such as a polylactide, polycaprolactone, polyglycolide, or copolymer thereof, in order to provide a more coherent solid implant or a composition with greater viscosity so as to hold it in place while it solidifies. Such thermoplastic polymers are disclosed in U.S. Pat. No. 4,938,763 to Dunn et al.

Optionally, a pore-forming agent can be included in the composition. The pore-forming agent can be any organic or inorganic, pharmaceutically-acceptable substance that is substantially soluble in water or body fluid, and will dissipate from the non-polymeric controlled release component material and/or the solid matrix of an implant into surrounding body fluid at the implant site. The pore-forming agent may preferably be insoluble in the organic solvent to form a uniform mixture with the non-polymeric material. The pore-forming agent may also be a water-immiscible substance that rapidly degrades to a water-soluble substance. In certain compositions, the pore-forming agent is combined with the non-polymeric material and organic solvent in admixture. Suitable pore-forming agents that can be used in the composition include, for example, sugars such as sucrose and dextrose, salts such as sodium chloride and sodium carbonate, polymers such as hydroxylpropylcellulose, carboxymethylcellulose, polyethylene glycol and polyvinylpyrrolidone, and the like. Solid crystals that will provide a defined pore size, such as salt or sugar, are preferred.

In other embodiments of the present invention, compositions are provided wherein the non-polymeric controlled release component is a liquid. The liquid non-polymeric material is preferably a high viscosity liquid carrier material (“HVLCM”), that is non-water soluble, and has a viscosity of at least 5,000 cP, (and optionally at least 10,000, 15,000; 20,000; 25,000 or even 50,000 cP) at 37° C. and does not crystallize neat under ambient or physiological conditions. The term “non-water soluble” refers to a material that is soluble in water to a degree of less than one percent by weight under ambient conditions. In the particular context of these liquid carrier materials, the term “non-polymeric” refers to esters or mixed esters having essentially no repeating units in the acid moiety of the ester, as well as esters or mixed esters having acid moieties wherein functional units in the acid moiety are repeated a small number of times (i.e., oligomers). Generally, liquid materials having more than five identical and adjacent repeating units or mers in the acid moiety of the ester are excluded by the term “non-polymeric” as used herein, but materials containing dimers, trimers, tetramers, or pentamers are included within the scope of this term. When the ester is formed from hydroxy-containing carboxylic acid moieties that can further esterify, such as lactic acid or glycolic acid, the number of repeat units is calculated based upon the number of lactide or glycolide moieties, rather than upon the number of lactic acid or glycolic acid moieties, where a lactide repeat unit contains two lactic acid moieties esterified by their respective hydroxy and carboxy moieties, and where a glycolide repeat unit contains two glycolic acid moieties esterified by their respective hydroxy and carboxy moieties. Esters having 1 to about 20 etherified polyols in the alcohol moiety thereof, or 1 to about 10 glycerol moieties in the alcohol moiety thereof, are considered non-polymeric as that term is used herein.

In certain compositions of the present invention, the HVLCM decreases in viscosity, in some cases significantly, when mixed with a solvent to form a low viscosity liquid carrier material (“LVLCM”) that can be administered using standard medical devices. The LVLCM composition is typically easier to place in the body than a HVLCM composition, because it flows more easily into and out of syringes or other implantation means. It also can easily be formulated as an emulsion. The LVLCM can have any desired viscosity, but its viscosity is generally lower than the corresponding HVLCM. As an example, viscosity ranges for the LVLCM of less than approximately 6,000 cP, less than approximately 4,000 cP, less than approximately 1,000 cP, or less than 200 cP, are typically useful for in vivo applications.

The particular non-polymeric HVLCM controlled release component used in the compositions of the invention can be one or more of a variety of materials. Suitable materials include nonpolymeric esters or mixed esters of one or more carboxylic acids. In a particular composition, the ester is formed from carboxylic acids that are esterified with a polyol having from about 2 to about 20 hydroxy moieties, and which may include 1 to about 20 etherified polyols. Particularly suitable carboxylic acids for forming the acid moiety of the ester of the HVLCM include carboxylic acids having one or more hydroxy groups, e.g., those obtained by ring opening alcoholysis of lactones, or cyclic carbonates or by the alcoholysis of carboxylic acid anhydrides. Amino acids are also suitable for forming esters with the polyol. In a particular composition, the ester or mixed ester contains an alcohol moiety having one or more terminal hydroxy moieties that have been esterified with one or more carboxylic acids obtained by alcoholysis of a carboxylic acid anhydride, such as a cyclic anhydride.

Nonlimiting examples of suitable carboxylic acids that can be esterified to form the HVLCM non-polymeric controlled release component include glycolic acid, lactic acid, ε-hydroxycaproic acid, serine, and any corresponding lactones or lactams, trimethylene carbonate, and dioxanone. The hydroxy-containing acids may themselves be further esterified through the reaction of their hydroxy moieties with additional carboxylic acid, which may be the same as or different from other carboxylic acid moieties in the material. Suitable lactones include, but are not limited to, glycolide, lactide, ε-caprolactone, butyrolactone, and valerolactone. Suitable carbonates include but are not limited to trimethylene carbonate and propylene carbonate.

In a particular embodiment, the HVLCM non-polymeric controlled release component may be sucrose acetate isobutyrate (SAIB) or some other ester of a sugar alcohol moiety with one or more alkanoic acid moieties.

In those compositions where the HVLCM non-polymeric controlled release component is mixed with a viscosity-lowering solvent to form a LVLCM, the solvents can be water soluble, non-water soluble, or water miscible, and can include, acetone, benzyl alcohol, benzyl benzoate, N-(betahydroxyethyl) lactamidebutylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, N-methyl-2-pyrrolidone, MIGLYOLs® (esters of caprylic and/or capric acids with glycerol or alkylene glycols, e.g., MIGLYOL® 810 or 812 (caprylic/capric triglycerides), MIGLYOL® 818 (caprylic/capric/linoleic triglyceride), MIGLYOL® 829 (caprylic/capric/succinic triglyceride), MIGLYOL® 840 (propylene glycol dicaprylate/caprate)), oleic acid, peanut oil, polyethylene glycol, propylene carbonate, 2-pyrrolidone, sesame oil, SOLKETAL ([±]-2,2-dimethyl-1,3-dioxolane-4-methanol), tetrahydrofuran, TRANSCUTOL® (diethylene glycol monoethyl ether, carbitol), triacetin, triethyl citrate, diphenyl phthalate, and combinations thereof. Additionally, if the composition is to be applied as an aerosol, e.g. for topical application, the solvent may be or may include one or more propellants, such as CFC propellants like trichlorofluoromethane and dichlorofluoromethane, non-CFC propellants like tetrafluoroethane (R-134a), 1,1,1,2,3,3,3-heptafluoropropane (R-227), dimethyl ether, propane, and butane.

Particularly suitable solvents and/or propellants include benzyl benzoate, benzyl alcohol, triacetin, triethyl citrate, dimethyl sulfoxide, ethanol, ethyl lactate, glycerol, glycofurol (tetraglycol), N-methyl-2-pyrrolidone, MIGLYOL® 810, polyethylene glycol, propylene carbonate, 2-pyrrolidone, and tetrafluoroethane. Other possible solvents include perfluorodecalin, perfluorotributylamine, methoxyflurane, glycerolformal, tetrahydrofurfuryl alcohol, diglyme, and dimethyl isosorbide.

In certain compositions, the selected solvent is at least water soluble, so that it will diffuse quickly into bodily fluids or other aqueous environment upon administration, causing the composition to coagulate and/or become more viscous. In another embodiments, the solvent is not completely miscible with water or bodily fluids so that diffusion of the solvent from the composition, and the corresponding increase in viscosity of the composition, are slowed.

In still further compositions provided according to the present invention, the composition includes a material that is not miscible with the HVLCM, such that when combined with the HVLCM singularly or in combination with a solvent for the HVLCM, the resulting composition forms an emulsion. Such emulsions may contain the HVLCM in the dispersed phase, such as in the case of SAIB/MIGLYOL® mixtures that are emulsified in water or glycerol, or they may contain the HVLCM as a component of the continuous phase, such as in the case of an aqueous solution that is emulsified in the HVLCM or a solution of the HVLCM in a water immiscible solvent.

D. Dosage Forms

The controlled release compositions of the present invention are in a general sense formed by the combination of the GnRH active agent with a suitable controlled release component, as described above, wherein the resulting composition provides for controlled release of the GnRH active to establish a sustained mean steady state plasma concentration (Cₛₛ) of the active of at least about 1.5 ng/mL for a period of at least about 48 hours when the composition is administered to a subject.

The particular formulation of the compositions of the present invention is within the general skill in the pharmaceutical arts, when applied using the teachings of the present specification and claims. Thus, suitable dosage forms can be provided establishing therapeutically effective plasma levels of the GnRH active for a period of at least about 48 hours in a subject after administration of the composition, wherein such plasma levels are substantially higher than those attained by the use of commercially available GnRH, or GnRH analog medicaments currently employed in the medical arts. Such dosage forms can then be used to establish a sustained mean Cₛₛ of the GnRH active on the order of at least about 1.5 ng/mL for a period of at least about 48 hours when the dosage form is administered to a subject, in some dosage forms, a sustained mean Cₛₛ of the GnRH molecule or GnRH analog on the order of at least about 2.0 ng/mL or more can be established, in other dosage forms at least about 2.5 ng/mL or more, and in yet further dosage forms, at least about 3.0 to 5.0 ng/mL or more. All of the dosage forms comprising the novel compositions of the present invention are capable of providing these high plasma levels for a period of at least about 48 hours in the subject after administration, in some cases, these levels can be established for a period of at least about a week or more or at least about 2 weeks or more, and in yet further cases these plasma levels are established for a period of at least about a month or more.

In certain preferred embodiments of the invention, the dosage form is produced using a combination of the GnRH active and a polymer controlled release component. Thus, suitable dosage forms can be manufactured when a hydrophobic polymer controlled release component is co-polymerized with a hydrophilic polymer, or monomers, to yield a suitable copolymer system, most preferably a block copolymer, or when the hydrophobic polymer component is blended with a hydrophilic polymer to yield a suitable blended polymer system. The polymer system can be produced using standard copolymerization techniques, such as graft copolymerisation, polycondensation and polyaddition, optionally with an appropriate catalyst. These techniques can be carried out in conventional manner with regard to time and temperature. Alternatively, the polymer system can be produced using standard blending techniques of polymers or blending of copolymers, again carried out in conventional manner with regard to time and temperature for the procedure.

Within the polymer system itself, the hydrophobic and hydrophilic components can be present in any suitable ratio, where the specific amount of each component is selected based on the relative degree of hydrophobicity or hydrophilicity of each component, respectively. Such dosage forms can be produced to exhibit monophasic or zero-order or near zero-order release kinetics of the GnRH active agent.

In certain preferred compositions, the polymer system used as the controlled release component is a copolymer or a polymer blend comprising a hydrophobic component selected from the group consisting of polyhydroxy acids, such as poly(lactide), poly(glycolide), poly(lactide-co-glycolide), poly(lactic acid), poly(glycolic acid), and poly(lactic acid-co-glycolic acid), polyanhydride, polyorthoester, polyetherester, polycaprolactone, polyesteramide, polyphosphazine, polycarbonate, polyamide, or any copolymer thereof.

In certain other preferred compositions, the polymer system used as the controlled release component is a copolymer or a polymer blend comprising a hydrophilic component selected from a poly (alkyleneglycol), polyvinyl pyrolidone (PVP), polyvinyl alcohol (PVOH), poly (alkyleneamine), poly (alkyleneoxide), or any copolymer thereof. In this regard, the hydrophilic component can be a poly (ethylene glycol) (PEG), and in certain cases, the hydrophilic component is a PEG having molecular weight of between about 700 Da and about 500 kDa.

In one specific embodiment, the polymer system used as the controlled release component is an AB block copolymer formed from poly (DL-lactide-co-glycolide) and PEG with a molecular weight of 750, wherein the PEG is present in the polymer system at about 1.25 wt %.

Once the suitable polymer system has been selected, the copolymerization or polymer blending step can be conducted either prior to incorporation of the GnRH active agent into the composition, or at the same time. The GnRH active agent is thus combined with the polymer controlled release component to produce the dosage form, using standard techniques. The GnRH active can be combined with the controlled release component such that it will be present in the compositions of the present invention in amounts ranging from about 0.1 wt % to about 80 wt % and higher, although the GnRH active agent will typically be present in an amount ranging from about 0.3 wt % to about 70 wt %, such as from about 10 wt % to 60 wt % or from about 20 wt % to about 55 wt %. The actual amount depends upon the activity of the selected GnRH active, the dose desired, the duration of release desired, the administration frequency and other variables. One skilled in the art will be able to ascertain effective amounts for selected GnRH actives by administration of the composition and observing the desired therapeutic, pharmacological or diagnostic effect. The exact amount of the GnRH active agent in the dosage form (composition) will thus be the amount necessary to achieve an effective concentration of the active agent in vivo, for a given period of time. This amount varies with the type of GnRH medicament used, the desired duration of the release, the target condition, desired administration frequency, the subject animal species and other factors. Preferably, the dosage forms will contain sufficient amounts of the GnRH active agent such that release of between about 0.10 ug/kg/day and 100 mg/kg/day will yield the desired effect. These parameters will be readily appreciated by the ordinarily skilled artisan upon reading the instant specification.

Depending upon the technique used to incorporate the GnRH active agent into the controlled release component and thus form the dosage forms of the invention, the GnRH active agent may be distributed uniformly within the controlled release component (e.g., a polymer system), or may be substantially encapsulated by the controlled release component. The GnRH active may further be incorporated into the composition using an appropriate solvent system, either aqueous or non-aqueous, or the GnRH active may be incorporated into the composition using a non-solvent process.

In addition to incorporation of the GnRH active agent with the controlled release component, the dosage forms may further include pharmaceutically acceptable excipients such as diluents, preservatives, solubilizers, emulsifiers and/or carriers needed for administration. The proportions in which a particular excipient may be present in the dosage form depends upon the purpose for which the excipient is provided and the identity of the excipient. The optimal final pharmaceutical formulation for a GnRH active agent of interest will be determined by one skilled in the art depending upon the route of administration and desired dosage. Exemplary pharmaceutical compositions are disclosed in Remington's Pharmaceutical Sciences (1990) Mack Publishing Co., 18th Ed., Easton, Pa.

In particular embodiments of the present invention, the above-described polymeric and non-polymeric controlled release components are used for manufacture of one or more compositions for controlled release of a GnRH molecule or GnRH analog, useful in the treatment or amelioration of the conditions the GnRH active agent is intended to treat.

The compositions of the present invention can be provided as one or more suitable dosage forms, depending upon the manner in which the compositions will be administered. In this regard, the dosage forms comprising the compositions of the invention may be administered by oral routes (e.g., as capsules such as hard capsules and soft capsules, solid preparations such as granules, tablets, pills, troches or lozenges, cachets, pellets, powders, particulates, microparticulates (and any other particulate form) and non-oral routes (e.g., as IM (intramuscular), SC (subcutaneous), transdermal, visceral, IV (intravenous), IP (intraperitoneal), intraarterial, intrathecal, intracapsular, intratumoral, perivascular, intracranial, intranasal, intrasinus, intrabladder, intravaginal, intraurethral, intrarectal, adventitial, injectable, pulmonary, inhalable, transmucosal, and other suitable forms). In certain embodiments, the dosage forms are intended for administration via implantation, and are thus configured as a shaped article, such as a sphere, rod, slab, film, fiber, needle, cylinder, sheet, tube, or any other suitable geometry including microparticles, microspheres, and/or microcapsules. Such dosage forms can be provided any suitable size and shape of implantable device for specialized locations, for example as a catheter, shunt, device for continuous subarachnoid infusion, feeding tube, solid implant, uterine implant, periurethral implant, splint, or stent (formed from, containing or coated with the composition). The dosage forms can be implanted at a desired site surgically, or using minimally invasive techniques employing trocars, catheters, etc. The implant can be implanted into any suitable tissue using standard techniques, such as implanted intradermally, subdermally, subcutaneously, intraperitoneally, intramuscularly, or intralumenally (e.g., intraarterially, intravenously, intravaginally, and the like). The dosage forms can alternatively be fabricated as part of a matrix, graft, prosthetic or coating. If an implantable dosage form is manufactured as a particulate, e.g., as a microparticle, microsphere or microcapsule, it can then be implanted into suitable tissue using a cannula, needle and syringe or like instrument to inject a suspension of the particles.

II. Methods of Manufacture

Tableting procedures, as well as formation of solutions, suspensions, emulsions, particles, microparticles, spheres, microspheres, films, etc. are all techniques well known in the pharmaceutical arts and within the skill of the general practitioner.

Methods for making fibrous polymeric dosage forms for delivery of active agents are also well known in the art. See, e.g., Cowsar and Dunn, Chapter 12 “Biodegradable and Nonbiodegradable Delivery Systems” pp. 145-162; Gibson, et al., Chapter 31 “Development of a Fibrous IUD Delivery System for Estradiol/Progesterone” pp. 215-226; Dunn, et al., “Fibrous Polymers for the Delivery of Contraceptive Steroids to the Female Reproductive Tract” pp. 125-146; Dunn, et al. (1985) “Fibrous Delivery Systems for Antimicrobial Agents” from Polymeric Materials in Medication ed. C. G. Gebelein and Carraher, Plenum Publishing Corporation, pp 47-59. Any of these known methods, and numerous other methods known in the art, may be employed in the practice of the present invention in order to produce fibrous dosage forms, comprising the compositions of the present invention and having the unique features described herein.

In addition, a variety of methods for processing polymer compositions by extrusion are described in Chris Rauwendaal (1994) “Polymer Extusion” Third Revised Edition, Carl Hanser Vertag, Munich, such as plasticating extrusion, where the polymer composition is fed to the extruder as a solid, and melt-fed extrusion where molten polymer is fed to the extruder. As used herein, the terms “extrusion” or “melt-spinning” encompasses all these methods of manufacture. In melt-spinning, a thermoplastic polymer is heated above its melting point, extruded through an orifice, and cooled to form a filament. In one particular method for producing dosage forms containing the compositions of the present invention, a selected GnRH active agent is mixed with a polymer controlled release component prior to extrusion and the mixture is then ground to form a feedstock for re-extruding the mixture to insure uniform mixing. Although generally formed in a geometry where the cross-section is a circle, such dosage forms can also be prepared with any other cross-sectional geometry, for example, an ellipsoid, a lobe, a square, or a triangle. The composition can also be formed into microparticles, sheets, films or coatings, using standard processing technology.

Suitable dosage forms may be prepared in a variety of sizes depending on the total dose of the GnRH active and the envisioned method and site of administration. In a certain composition, the dosage form is a monolithic rod with an overall diameter between 0.05 and 5.0 mm. For subcutaneous administration in humans, an overall diameter of between 1.0 and 4.0 mm may be more preferred. The length of the device is typically between about 0.3 cm and 10 cm. For subcutaneous implantation, a more preferred length is between about 0.3 cm and 3.0 cm.

Drawing may used to produce extruded dosage forms, such as methods where the composition is passed around two or more sets of godets that are operated at progressively faster speeds as the composition passes further down the line. The composition may pass through heated ovens between the godets so that the temperature can be carefully controlled to further influence the crystallinity of any controlled release components and excipients. Drawing may also be used to control the final diameter of the dosage form.

Because of the basic structure of dosage forms prepared by a continuous extrusion process, they can be provided in any length that is convenient for handling. If the composition is sufficiently flexible, it can be wound onto a spool or into a coil and held in this way prior to cutting. Alternatively, the extruded composition can be collected as shorter lengths of perhaps a few centimeters or meters and held prior to cutting. It is also possible to cut the extruded composition to the finished dosage form length as it is produced using a flywheel type of cutter that is situated just downstream of the die.

The amount of the GnRH active agent to be incorporated and the amount used in the process will vary depending upon the particular agent, the desired effect of the active agent at the planned release levels, and the time span over which the agent should be released. Any of the above-described processes can be used to incorporate more than one GnRH active agent into a controlled release composition.

III. Methods of Use

It is an object of the invention to provide methods for establishing therapeutically effective plasma levels of a GnRH molecule or GnRH analog for about 48 hours or more in a subject. The methods generally entail administering any one of the above-described controlled release compositions (as a suitable dosage form) to the subject such that, after administration, the administered composition provides a sustained mean steady state plasma concentration (Cₛₛ) of the GnRH molecule or GnRH analog of at least about 1.5 ng/mL for a period of at least about 48 hours in the subject.

It is more particularly an object of the present invention to provide a method suitable for establishing therapeutically effective plasma levels of a GnRH molecule or GnRH analog for a period of at least about 48 hours in a subject after administration of the composition, wherein such plasma levels are substantially higher than those attained by the use of commercially available GnRH, or GnRH analog medicaments currently employed in the medical arts. Accordingly, the methods of the present invention can be used to establish a sustained mean Cₛₛ of the GnRH molecule or GnRH analog on the order of at least about 1.5 ng/mL for a period of at least about 48 hours after the composition is administered to a subject, in some particular methods, a sustained mean Cₛₛ of the GnRH molecule or GnRH analog on the order of at least about 2.0 ng/mL or more can be established, in other methods at least about 2.5 ng/mL or more, and in yet further methods, at least about 3.0 to 5.0 ng/mL or more. All of the novel methods of the present invention are capable of providing these high plasma levels for a period of at least about 48 hours in the subject after administration. In some compositions, these levels can be established for a period of at least about a week or more or at least about 2 weeks or more, and in yet further compositions these plasma levels are established for a period of at least about a month or more.

The controlled release compositions, provided in the form of one or more dosage forms in accordance with the invention, can be administered using any suitable procedure. Depending upon the particular GnRH active agent to be administered, the selected dosage form (size, shape, etc.) and the selected site of administration, the controlled release compositions can be administered or implanted using minimally invasive procedures at a site where delivery is desired. These procedures can include implantation using trocars or catheters, injection using standard needle and syringes (of, e.g., powders, particles, microparticles, microspheres, microcapsules), ingrafting or surgical or non-surgical placement (of, e.g., a matrix, graft, prosthetic or coating), inhalation (of, e.g., powders or particulates), and the like. The compositions are designed so that the GnRH active agent is released in the desired dosage over a defined period of time. The compositions may further be designed so that they degrade during and after controlled release of the active agent is achieved.

In certain aspects of the invention, the controlled release composition is provided and administered as a single dosage form. For example, the composition can be provided as an implantable solid dosage form such as a rod. In other aspects, the composition is provided and administered as a plurality of dosage forms. For example, the compositions of the invention can be provided as a combination of an implantable solid dosage form and an injectable depot. In certain other aspects, the composition is provided as a single dosage form that is administered as a single dosage unit, that is, a single dosage form is used to provide the recited sustained mean steady state plasma concentrations of the GnRH active. For example, a single solid implantable dosage form such as a rod can be administered to a subject to provide the desired pharmacokinetics of the present invention. In other instances, multiple dosage units of a single dosage form can be administered to provide the recited sustained mean steady state plasma concentrations of the GnRH active, such as wherein a plurality (two or more) of solid implantable dosage forms are administered either simultaneously, concurrently, or sequentially to provide the desired pharmacokinetics of the present invention.

In yet further aspects of the invention, multiple dosage forms, each representing a single dosage unit, can be administered either simultaneously, concurrently, or sequentially to provide the desired pharmacokinetics of the present invention. Whenever multiple dosage forms and/or units are administered, the actual dose of the GnRH active in each form or unit can be the same or different. In this way, any desired sustained mean steady state plasma concentration of the GnRH active can be achieved in a given subject by way of administering a single dosage form and/or dosage unit of sufficient dose, or by combining a plurality of dosage forms and/or units containing the same or different dose of the GnRH active to tailor a specific dose sufficient to establish the desired plasma concentration in a given subject.

In one particular embodiment, the controlled release composition is provided in the form of multiple extruded solid implant rods, having a GnRH active loading of about 30 wt % relative to the total weight of the composition. The implants are administered subcutaneously at substantially the same or different sites on the subject using a trocar style administration device. The implants are left in place to provide a sustained mean Cₛₛ of the GnRH molecule or GnRH analog on the order of at least about 1.5 ng/mL for a period of at least about 48 hours after the implants are administered to a subject. In some subjects, the method is carried out to provide a sustained mean Cₛₛ of the GnRH molecule or GnRH analog on the order of at least about 2.0 ng/mL or more, in other subjects at least about 2.5 ng/mL or more, and in yet further subjects, at least about 3.0 to 5.0 ng/mL or more. All of these methods are capable of being performed to provide high plasma levels for a period of at least about 48 hours in the subject after administration, in some cases, these levels can be established for a period of at least about a week or more or at least about 2 weeks or more, and in yet further cases these plasma levels are established for a period of at least about a month or more, at least about 2 months or more, or even at least about 3 months or more.

Any of the methods of the invention can be carried out to treat a variety of diseases and conditions, e.g., to treat hormone-dependent cancers (such as prostate cancer); to treat endometriosis; to treat early puberty, to control estrogen production; to treat fertility conditions; and the like.

In one embodiment, a controlled release composition is formulated to include a GnRH active as one or more solid implant dosage form. The composition is then administered to a subject in order to target blood level, production, function, or activity of a gonadotrophin LH or FSH similar to that occurring at or near the time of greatest reproductive function in the subject, which in humans corresponds to 18 to 35 years of age. For example, a normal blood level of LH around this time is approximately 0-10.0 mIU/mL for males and approximately 0.4-92.9 mIU/mL for females (which fluctuates with reproductive cycle). A normal blood level of FSH around this time is approximately 2.0-22.6 mIU/mL for males and approximately 2.9-29.5 mIU/mL for females (which also fluctuates with reproductive cycle). Administration of the GnRH active implant is suitable to alter the blood level, production, function, or activity of a gonadotrophin LH or FSH to achieve the desired level(s).

In another embodiment, a controlled release composition is formulated to include a GnRH active as one or more solid implant dosage form. The composition is then administered to a subject in order to the target blood level, production, function, or activity of LH or FSH to levels that are undetectable or nearly undetectable. For example, a blood level of 0.7 mIU/mL for both LH and FSH is currently undetectable in a clinical laboratory.

In another embodiment of the invention, a controlled release composition is formulated to include a GnRH active as one or more solid implant dosage form. The composition is then administered to a subject in order to the target blood level, production, function, or activity of LH or FSH to levels as low as possible without unacceptable adverse side effects. An unacceptable adverse side effect is an adverse side effect that, in the reasonable judgment of one of ordinary skill in the art, has costs that outweigh the benefits of treatment.

In the practice of these and other related methods, the subject's blood level, production, function, or activity of LH or FSH may be periodically monitored and the combinations, quantities, and dosage regimens of the LH/FSH-inhibiting agents may be titrated or varied in order to achieve the target blood level, target production, target function or target activity of LH and FSH. In a particularly preferred embodiment, the dosage for a GnRH active, for example leuprolide acetate, may be between approximately 0.01 mcg/kg/hour and approximately 100 mg/kg/day, or other schedules that will be apparent to one of ordinary skill in the art, in light of this specification. In these methods, the subject may initially be administered a low dose, for example approximately 0.01 mcg/kg/hour. After approximately two weeks, LH and FSH blood levels may be measured. If LH and FSH bloods levels are still higher than the target, then the dose may be increased (for example by 0.1 mcg/kg/hour). This titration can be repeated until the blood level, production, function or activity of LH or FSH reaches the desired target blood level, production, function, or activity for LH or FSH, as set forth above.

For example, a 30 mg time-released dose of leuprolide acetate can be administered to an adult male subject. The leuprolide acetate active agent is provided in a biodegradable polymer controlled release component to supply a polymeric dosage form for controlled release of the GnRH active. The polymer component is a copolymer or a polymer blend comprising a hydrophobic component and a hydrophilic component and the polymer system does not form a hydrogel when contacted with, or immersed in an aqueous system, for example when the composition is implanted in the subject. The leuprolide acetate active agent is incorporated within the polymer controlled release component to provide for controlled release of the agent from the composition. When the composition is administered to the subject, for example, when it is implanted, the composition releases the GnRH active agent in a controlled fashion to provide a sustained mean C, of the active agent on the order of at least about 1.5 ng/mL for a period of at least about 48 hours after the composition is administered to a subject. Release of the GnRH active preferably occurs without a lag period, or with a minimal lag period. In this manner, the leuprolide can be gradually released over a period of several months. After a period of two weeks, the subject's blood level of LH may be undetectable and the subject's blood level of FSH may be approximately 5 mIU/mL.

In another example, a dose of 1.88 mg time-released dose of leuprolide acetate can be administered to a subject. The leuprolide acetate active agent is present in a composition formed with a biodegradable polymer controlled release component to provide for controlled release of the GnRH active agent. The polymer is a copolymer or a polymer blend comprising a hydrophobic component and a hydrophilic component and the polymer system does not form a hydrogel when contacted with, or immersed in an aqueous system, for example when the composition is implanted in the subject. When the composition is administered to the subject, for example, when it is implanted, the GnRH active agent is released in a controlled fashion to provide a sustained mean Cₛₛ of the active agent on the order of at least about 1.5 ng/mL for a period of at least about 48 hours after the composition is administered to a subject. Release of the GnRH active preferably occurs without a lag period, or with a minimal lag period. In this manner, the leuprolide can be gradually released over approximately one month, and is expected to reduce LH and FSH blood levels to undetectable levels in the subject. It will be apparent to one of ordinary skill in the art, in light of this specification, that in order to achieve this target, the dosage of the leuprolide active agent will vary from subject to subject in light of factors such as age, gender, body weight, diet, the disease being treated, the progression of the disease, and other drugs being administered.

Modifications and variations of the present invention will be obvious to those skilled in the art and are intended to come within the scope of the appended claims.

1. A controlled release composition, comprising:
(a) a GnRH molecule or GnRH analog; and
(b) a controlled release component for controlling release of the GnRH molecule or GnRH analog from the composition, wherein said composition is capable of providing a sustained mean steady state plasma concentration (Cₛₛ) of the GnRH molecule or GnRH analog of at least about 1.5 ng/mL for a period of at least about 48 hours when said composition is administered to a subject.

2. The composition of claim 1 wherein said composition is provided in a single dosage form.

3. The composition of claim 2 wherein said composition is further provided as a single dosage unit.

4. The composition of claim 2 wherein said composition is further provided as multiple dosage units.

5. The composition of claim 1 wherein said composition is provided in a plurality of dosage forms.

6. The composition of claim 1, wherein the composition is provided as at least one implant dosage form.

7. The composition of claim 6, wherein the implant dosage form is a solid.

8. The composition of claim 7 wherein the implant dosage form is a fiber, needle, rod, sheet, film, particle or microparticle.

9. The composition of claim 6 wherein said implant is monolithic.

10. The composition of claim 6, wherein the implant dosage form is injectable.

11. The composition of claim 10, wherein the implant dosage form is injectable to form a depot.

12. The composition of claim 10, wherein the implant dosage form is injectable to form a solid or semi-solid implant.

13. The composition of claim 10 wherein the implant dosage form is a sphere or a microsphere.

14. The composition of claim 1, wherein the controlled release component comprises a polymer material.

15. The composition of claim 14, wherein the GnRH molecule or GnRH analog is distributed uniformly within the polymer material.

16. The composition of claim 14, wherein the GnRH molecule or GnRH analog is coated with the polymer material.

17. The composition of claim 14, wherein the polymer material of the controlled release component comprises at least one is material selected from the group consisting of polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polycaprolactone, polyesteramides, polyphosphazines, polycarbonates, polyamides, and copolymers thereof.

18. The composition of claim 1, wherein the controlled release component comprises a non-polymer material.

19. The composition of claim 1, wherein the controlled release component provides for release of the GnRH molecule or GnRH analog with linear or near zero order release kinetics.

20. The composition of claim 1, wherein the GnRH molecule or GnRH analog is released from the composition without a significant initial burst.

21. The composition of claim 20, wherein less than about 30% of the GnRH molecule or GnRH analog is released from the composition within about 24 to 48 hours of administration to a subject.

22. The composition of claim 1, wherein the GnRH molecule or GnRH analog is present in the composition in an amount of at least about 20 wt % relative to the total weight of the composition.

23. The composition of claim 1, wherein the GnRH molecule or GnRH analog is present in the composition in an amount of at least about 30 wt % relative to the total weight of the composition.

24. The composition of claim 1, wherein the total amount of the GnRH molecule or GnRH analog in the composition is between about 5 and 20 mg.

25. A composition according to claim 1 capable of providing a sustained mean steady state plasma concentration (Cₛₛ) of the GnRH molecule or GnRH analog of at least about 1.5 ng/mL for a period of at least about 1 week when said composition is administered to a subject.

26. A composition according to claim 1 capable of providing a sustained mean steady state plasma concentration (Cₛₛ) of the GnRH molecule or GnRH analog of at least about 1.5 ng/mL for a period of at least about 2 weeks when said composition is administered to a subject.

27. A composition according to claim 1 capable of providing a sustained mean steady state plasma concentration (Cₛₛ) of the GnRH molecule or GnRH analog of at least about 1.5 ng/mL for a period of at least about 1 month when said composition is administered to a subject.

28. A composition according to claim 1 capable of providing a sustained mean steady state plasma concentration (Cₛₛ) of the GnRH molecule or GnRH analog of at least about 2.0 ng/mL for a period of at least about 48 hours when said composition is administered to a subject.

29. A composition according to claim 1 capable of providing a sustained mean steady state plasma concentration (Cₛₛ) of the GnRH molecule or GnRH analog of at least about 2.5 ng/mL for a period of at least about 48 hours when said composition is administered to a subject.

30. A composition according to claim 1 capable of providing a sustained mean steady state plasma concentration (Cₛₛ) of the GnRH molecule or GnRH analog of at least about 5.0 ng/mL for a period of at least about 48 hours when said composition is administered to a subject.

31-60. (canceled)

61. A method for providing therapeutically effective plasma levels of a GnRH molecule or GnRH analog for about 48 hours or more in a subject, said method comprising administering the controlled release composition of claim 1 to the subject, whereby after administration, said composition provides a sustained mean steady state plasma concentration (Cₛₛ) of the GnRH molecule or GnRH analog of at least about 1.5 ng/mL for a period of at least about 48 hours in the subject.

62. A method for providing therapeutically effective plasma levels of a GnRH molecule or GnRH analog for about 1 week or more in a subject, said method comprising administering the controlled release composition of claim 25 to the subject, whereby after administration, said composition provides a sustained mean steady state plasma concentration (Cₛₛ) of the GnRH molecule or GnRH analog of at least about 1.5 ng/mL for a period of at least about 1 week in the subject.

63. A method for providing therapeutically effective plasma levels of a GnRH molecule or GnRH analog for about 2 weeks or more in a subject, said method comprising administering the controlled release composition of claim 26 to the subject, whereby after administration, said composition provides a sustained mean steady state plasma concentration (Cₛₛ) of the GnRH molecule or GnRH analog of at least about 1.5 ng/mL for a period of at least about 2 weeks in the subject.

64. A method for providing therapeutically effective plasma levels of a GnRH molecule or GnRH analog for about 1 month or more in a subject, said method comprising administering the controlled release composition of claim 27 to the subject, whereby after administration, said composition provides a sustained mean steady state plasma concentration (Cₛₛ) of the GnRH molecule or GnRH analog of at least about 1.5 ng/mL for a period of at least about 1 month in the subject.

65. A method for providing therapeutically effective plasma levels of a GnRH molecule or GnRH analog for about 48 hours or more in a subject, said method comprising administering the controlled release composition of claim 28 to the subject, whereby after administration, said composition provides a sustained mean steady state plasma concentration (Cₛₛ) of the GnRH molecule or GnRH analog of at least about 2.0 ng/mL for a period of at least about 48 hours in the subject.

66. A method for providing therapeutically effective plasma levels of a GnRH molecule or GnRH analog for about 48 hours or more in a subject, said method comprising administering the controlled release composition of claim 29 to the subject, whereby after administration, said composition provides a sustained mean steady state plasma concentration (Cₛₛ) of the GnRH molecule or GnRH analog of at least about 2.5 ng/mL for a period of at least about 48 hours in the subject.

67. A method for providing therapeutically effective plasma levels of a GnRH molecule or GnRH analog for about 48 hours or more in a subject, said method comprising administering the controlled release composition of claim 30 to the subject, whereby after administration, said composition provides a sustained mean steady state plasma concentration (Cₛₛ) of the GnRH molecule or GnRH analog of at least about 5.0 ng/mL for a period of at least about 48 hours in the subject.
